(19) United States
(12) Patent Application Publication
(10) Pub. No.: US 2017/0360592 A1
(43) Pub. Date: Dec. 21, 2017

(54) OSTOMY APPLIANCE

(71) Applicant: TENCAR INC., Batavia, NY (US)

(72) Inventor: Georgann M. Carrubba, Batavia, NY (US)

(73) Assignee: TENCAR INC., Batavia, NY (US)

(21) Appl. No.: 15/696,967

(22) Filed: Sep. 6, 2017

Related U.S. Application Data
(63) Continuation-in-part of application No. 15/419,695, filed on Jan. 30, 2017.
Application No. 15/419,695 is a continuation-in-part of application No. 14/617,460, filed on Feb. 9, 2015, now Pat. No. 9,757,270.
Application No. 14/617,460 is a continuation-in-part of application No. 14/924,496, filed on Oct. 27, 2015.
Application No. 14/924,496 is a continuation-in-part of application No. 14/617,460, filed on Feb. 9, 2015, now Pat. No. 9,757,270.

(51) Int. Cl. A61F 5/441; A61F 5/44; A61F 5/443; A61F 5/448

(52) U.S. Cl. CPC A61F 5/441; A61F 5/448; A61F 5/4401; A61F 5/4404; A61F 5/443

(57) ABSTRACT

An ostomy appliance having a releasable receptacle for concealing an ostomy bag where the receptacle is asymmetrical and includes an enlarged cavity portion and expandable portion located below the stoma which provides additional area into which the bag may unroll as waste collects therein. In an alternate embodiment there is no bag and waste collects directly into the receptacle.

### BACKGROUND OF THE INVENTION

The present invention relates to ostomy appliances. More particularly, the present invention relates to an ostomy appliance which is designed to provide a high level of discreteness to the user on an as-needed basis.

An “ostomy” is the generic term for a surgical procedure such as the ileostomy, colostomy and urostomy which leave the patient with a digestive waste opening formed by attaching the patient's intestine (small intestine in the case of an ileostomy; large intestine in the case of a colostomy) to the perimeter of an incision made in the abdominal wall. This part of the intestine is surgically opened to form what is called the “stoma” wherethrough digestive waste exits the body. The procedure may be reversible or irreversible depending on the circumstances. It is estimated that up to 750,000 Americans have an ostomy.

A digestive waste collection bag is attached about the stoma to collect the waste exiting therefrom. The collection bag and associated body attachment parts are commonly referred to as the ostomy appliance or assembly. Ostomy appliances are offered in single piece and two piece systems. In the single piece system, the bag has an opening which is aligned with an opening in and then permanently fixed to a disc or flange which has an adhesive surface opposite the bag which secures the flange and bag to the body about the stoma. In the two-piece system, the bag is detachably secured to the flange, commonly via a snap ring on the flange which fits into a cooperatively formed groove attached to the perimeter of the bag opening. In either the single or two piece systems, the bag typically includes an open end opposite the flange end which may be alternately open and closed using a clamp. The bag is clamped closed during use and opened to empty the waste contents from the bag. The bag may be cleaned and reused or discarded with a new bag being attached to the flange.

Users of ostomy appliances often complain about the inconveniences of the appliance including lack of discreteness. For example, there is no control over when waste deposits into the bag which may thus fill and noticeably expand beneath the clothing at inconvenient times, inevitably causing embarrassment to the user. Attempts to address this problem have been at best a minor improvement while others appear to pose the threat of actual physical harm to the user by applying a positive pressure against the stoma.

One example of an ostomy appliance which addresses the above drawbacks in the prior art is shown and described in commonly owned U.S. Pat. No. 8,657,799, the disclosure of which is incorporated herein by reference. The '799 patent provides an ostomy appliance which is compact and has a receptacle portion which accommodates a certain amount of waste before it must be opened and emptied. For users that desire to wear the ostomy appliance a bit longer than the '799 receptacle may allow, there remains a need for an ostomy appliance having a receptacle designed to accommodate a bit more waste. This would allow the user to wear the appliance for a longer time prior to opening and emptying yet still provide enhanced discreteness during periods of physical activity and close personal encounters, all without application of potentially dangerous pressures on the stoma as occurs in the prior art.

### SUMMARY OF THE INVENTION

The present invention provides an ostomy appliance and method which includes a cup-shaped receptacle having an attachment mechanism about the open perimeter thereof which may be removably secured to a mating attachment mechanism (e.g., an ostomy flange) secured to the adhesive patch which includes a central opening for locating about the stoma opening on the user's body. The bag includes a ring element at the open stoma-facing end thereof which removably attaches to a mating ring element located around the stoma opening and radially inwardly of the receptacle attachment mechanism on the patch. When it is desired to contain the bag for increased discreteness, the user gathers or rolls the empty bag up upon itself into a rolled position, positions the receptacle over the bag and then secures the cooperative attachment mechanism together thereby securing the receptacle to the ostomy flange with the bag enclosed therein in the rolled condition.

The receptacle may have one or more vent holes formed therein to allow gas to escape therefrom. A vent cap may be provided which is normally closed but will automatically open upon a rise in pressure inside the receptacle to allow the egress of gas from the receptacle. Once the pressure is relieved the vent cap automatically closes. A covering may be placed over the receptacle to provide further discreteness and comfort to the user.

The receptacle includes an enlarged internal cavity which, when attached to the ostomy flange on the user's body, preferably extends downwardly in line with the length of the body toward the user's feet. This enlarged cavity portion of the receptacle provides a portion within which the bag may deploy (e.g., unroll) as the bag fills with the user's waste. This particular embodiment thus allows the receptacle to remain in place for a longer time than a receptacle that has a smaller receptacle cavity size. In a preferred embodiment, the receptacle is asymmetrically shaped with the smaller side of the receptacle located above the stoma. This asymmetrical shape helps minimize the size of the receptacle while still proving added cavity portion where it is most useful. More particularly, the side of the receptacle which is smaller extends above the stoma where waste is not normally directed and the enlarged area is located on the opposite side of the receptacle where the waste and bag are directed by gravity when the user is sitting upright or standing.

When the bag is held in the receptacle, waste will deposit therein in the usual manner. As waste enters the bag, the bag will begin to expand and unroll slightly within the confines of the receptacle. There is thus no positive pressure being applied to the stoma and waste is allowed to naturally exit the user's stoma and enter the bag. It is not intended that the receptacle be in place over the bag for more than a few hours at a time and it is therefore not expected that waste will be prevented from naturally exiting the stoma for any potentially harmful extended period of time. Rather, the receptacle is only intended to be in place during times when the user would like an increased level of discreteness and control over waste evacuation into the bag. Once the bag is full and/or the receptacle is no longer needed, the user simply detaches the receptacle from the flange whereupon the bag is allowed to fully unroll and is either left to completely fill or emptied and cleaned or replaced with a new bag, as desired.

In another embodiment, the bag is not used and waste collects directly into the receptacle which may be removed, emptied of waste, cleaned and reattached as desired. This provides an option to the user who may not want to use a bag or when their bag supply has run out.

In a further embodiment of the present invention, the receptacle may be configured to include an expandable portion configured to expand the enlarged cavity portion still further. In this manner, a user may enjoy even longer periods of time between cleaning or replacing of the bag or receptacle with minimized concern for accidents or blockages due to restriction of the natural exiting of waste through the stoma.

### BRIEF DESCRIPTION OF THE DRAWING

The above-mentioned and other features and advantages of this invention, and the manner of attaining them, will become apparent and be better understood by reference to the following description of the invention in conjunction with the accompanying drawing, wherein:

FIG. 1 is a perspective view of an embodiment of the inventive ostomy device;

FIG. 2 is a side elevational view of the ostomy device of FIG. 1 showing the covering in position over the ostomy bag container with parts thereof shown in section;

FIG. 3 is an exploded perspective view of the ostomy device absent the ostomy bag;

FIG. 4 is a perspective view showing the ostomy bag attached to the stoma flange with the bag in the unfolded position;

FIG. 5 is the view of FIG. 4 showing the ostomy bag in the folded condition and the ostomy bag container in portioned relation thereto;

FIG. 6 is the view of FIG. 5 showing the ostomy bag container attached to the stoma flange with the folded bag showing in dotted lines inside the container;

FIG. 7 is a side elevational view of an alternative embodiment of an ostomy device with parts thereof shown in section;

FIG. 8 is a side elevational view of another alternative embodiment of an ostomy device with parts thereof shown in section;

FIG. 9 is a perspective view of another alternative receptacle to be used within the embodiment of the inventive ostomy device of FIG. 1; and

FIG. 10 is a side elevational view of the alternative receptacle of FIG. 9 with parts thereof shown in section generally along line 10-10 shown in FIG. 9.

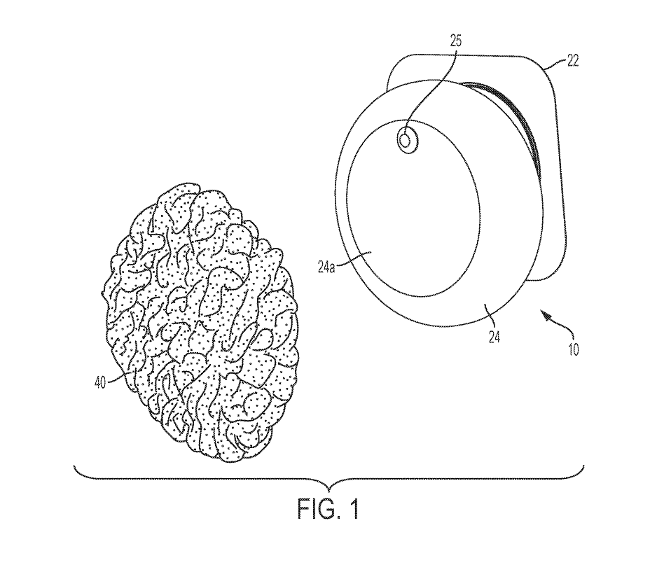

### DETAILED DESCRIPTION OF A PREFERRED EMBODIMENT

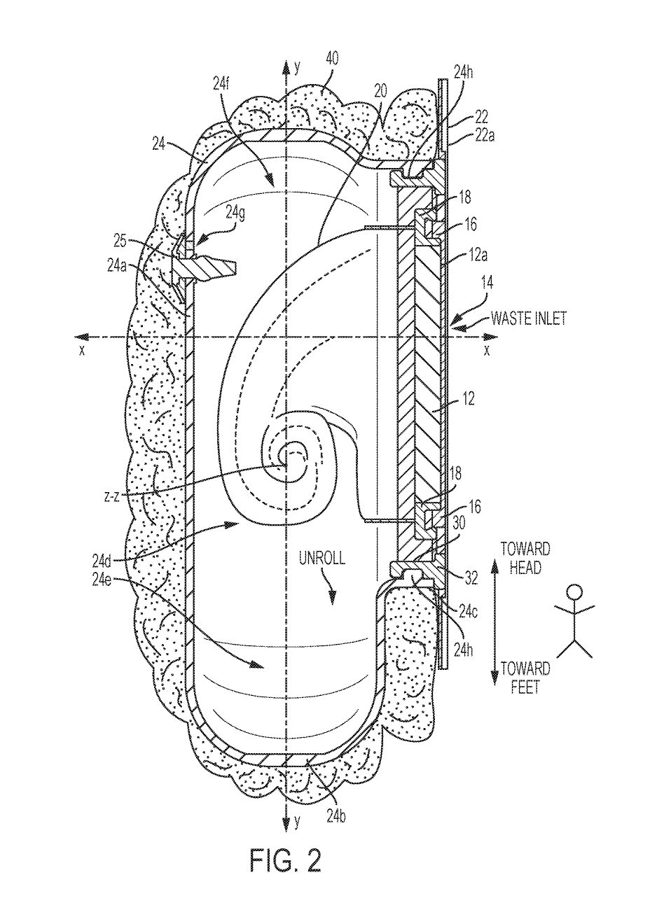

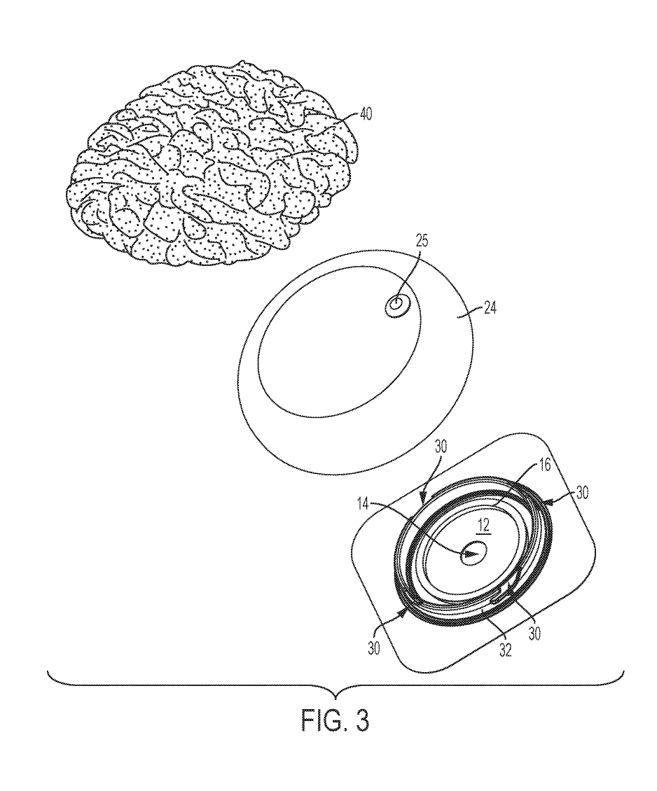

Referring now to the drawing, there is seen in the various figures an ostomy appliance according to one embodiment of the invention indicated generally by the reference numeral 10. Ostomy appliance 10 includes a disc 12 having a central opening 14 for aligning with and positioning over a user's stoma (not shown). Disc 12 includes an adhesive surface 12a (see FIG. 2) for securing the disc about the stoma body tissue. Release paper (not shown) protects the adhesive surface 12a until time of use whereupon the user removes the release paper and adheres the disc surface 12a onto the stoma tissue with the opening of the stoma aligned with the disc opening 14.

A ring-shaped groove element 18 is attached to the perimeter of a first opening 20a of waste collection bag 20 and a ring-shaped flange 16 is affixed to disc 12 such that the bag may be releasably attached to the disc 12. Skirt 22 extends outwardly from ring-shaped flange 16 and includes an adhesive surface 22a for adhering to the user's body tissue which surrounds the stoma. A release paper (not shown) may protect the adhesive surface 22a until time of use whereupon the user removes the release paper and adheres the skirt 22 to the user's body while also adhering the disc adhesive surface 12a to the adjacent stoma tissue.

A cup-shaped receptacle 24 is provided for times when the user desires an increased level of discreteness. Receptacle 24 includes a bottom wall 24a and a side wall 24b terminating in a top perimeter edge 24c all defining an interior cavity 24d. The shape of the receptacle is such that side wall 24b forms an enlarged cavity portion 24e which extends downwardly in line with the length of the body toward the legs when attached to the user in the intended manner. This enlarged cavity portion 24e of the receptacle provides more portion within which the bag 20 may fit and begin to expand or unroll as the bag fills with the user's waste. Receptacle 24 may be fabricated from a rigid yet flexible material, such as natural or synthetic rubber or a molded thermoplastic material, including without limitation, nylon, polypropylene, polyethylene and copolymers thereof. This particular embodiment thus allows the receptacle to remain in place for a longer time than a receptacle that is more compact.

As seen best in FIG. 2, receptacle 24 is asymmetrically shaped about a horizontal axis x-x (relative to vertically standing user) extending through the center of opening 14 which is substantially concentrically placed over the user's stoma. The receptacle smaller cavity portion 24f is located above axis x-x and the stoma (toward user's head) and the enlarged cavity portion 24e is located below axis x-x and the stoma (toward the user's feet). This asymmetrical shape helps minimize the overall size of the receptacle in that the smaller cavity portion 24f is located above the stoma where waste does not usually get directed and the enlarged cavity portion 24e is located below the stoma where the waste and bag 20 are directed within the confines of the receptacle 24 due to gravity when the user is sitting upright or standing. In this regard, it is preferred that the user roll bag 20 up about an axis z-z which extends in the horizontal plane of and perpendicular to axes x-x and longitudinal axis y-y. As such, the bag 20 will be able to freely unroll in the direction of enlarged cavity portion 24e along and parallel to vertical longitudinal axis y-y as receptacle 24 is filled with waste. The rigidity of receptacle 24 may allow bag 20 to occupy enlarged cavity portion 24e while also constraining the length of the opening bag in the y-y axis. In this manner, a user may be comforted knowing that bag 20 may fill without resulting in unsightly bulges or overly elongated bags.

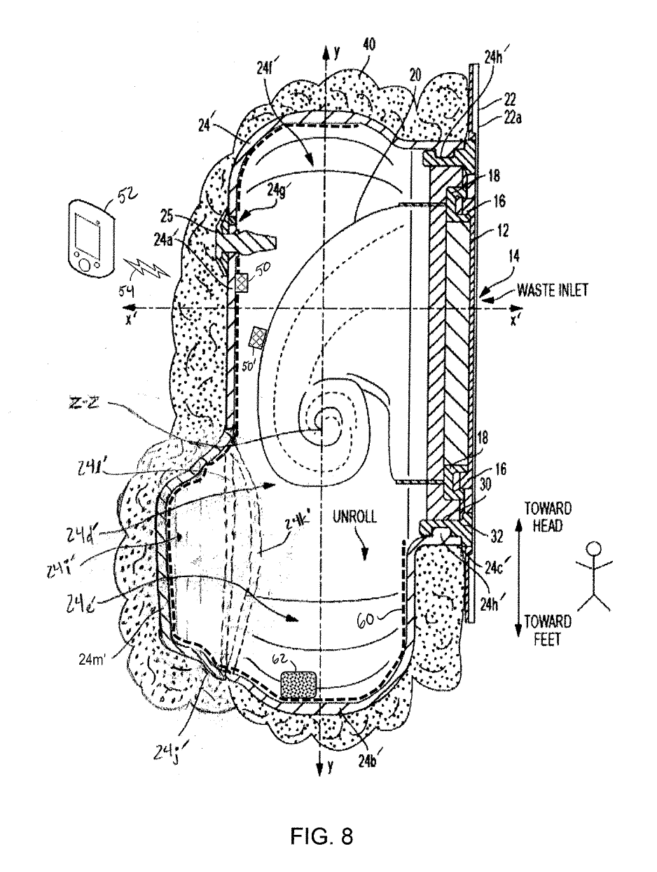

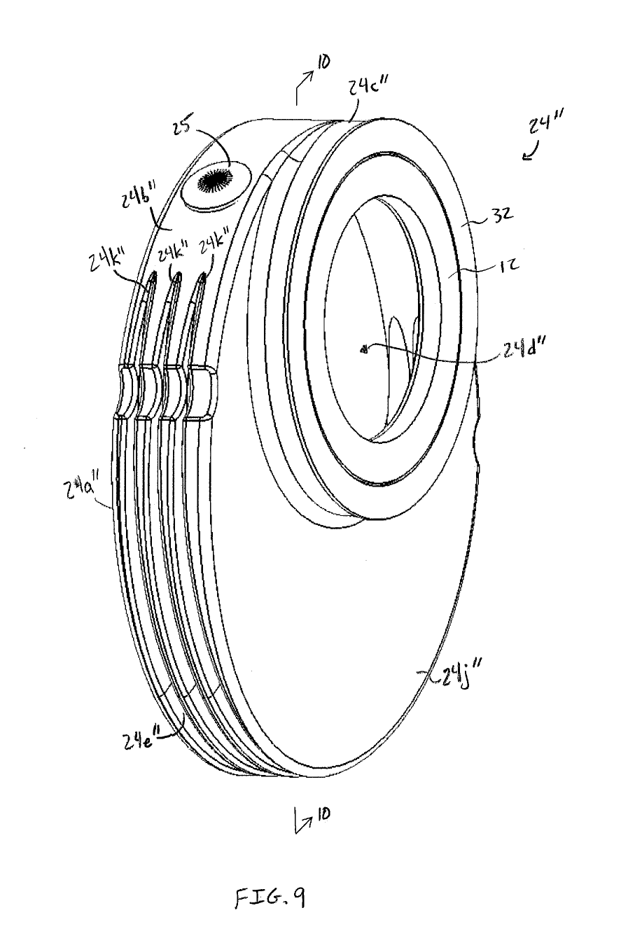

With reference to FIG. 8, an alternative cup-shaped receptacle 24′ may be provided for times when the user desires a still greater increased level of discreteness. To that end, receptacle 24′ includes a bottom wall 24a′ and a side wall 24b′ terminating in a top perimeter edge 24c′ all defining an interior cavity 24d′ as described above with regard to receptacle 24. Also as described above, the shape of the receptacle is such that side wall 24b′ forms an enlarged cavity portion 24e′ which extends downwardly in line with the length of the body toward the legs when attached to the user in the intended manner. This enlarged cavity portion 24e′ of the receptacle provides more space within which the bag 20 may fit and begin to expand or unroll as the bag fills with the user's waste. Receptacle 24′ includes the further provision of an expandable cavity portion 24i′ defined by expandable wall 24j′ of bottom wall 24a′. Expandable wall 24j′ may be conterminous with or nearly coterminous with sidewall 24b′ so as to promote entry of bag 20 within expandable cavity portion 24i′. Receptacle 24′ may thus provide even more space within which bag 20 may fit and begin to expand or unroll.

In a further aspect of the present invention, expandable wall 24j′ may be constructed to expand/contract through accordion-style folding of expandable wall 24j′. However, to minimize potential for contamination and increase ease in cleaning, expandable wall 24j′ may be fabricated from an elastic material, such as a synthetic elastomer or copolymers thereof, whereby expandable wall 24j′ may expand and contract without trapping waste within folds of the material. In should be noted that while latex may be a suitable material for expandable wall 24j′, care should be taken due to the potential for latex allergies. To that end, expandable wall 24j′ should be easy to clean with conventional household cleaners while also being hypo-allergenic.

When in its resting state, expandable wall 24j′ may extend within enlarged cavity portion 24e′ as indicated generally by dashed lines 24k′ or may lay coplanar with bottom wall 24a′ as generally indicated by dashed lines 24l′. In either event, expandable wall 24j′ is configured to expand outwardly 24m′ to form expandable cavity portion 24i′ upon filling with waste. Expandable wall 24j′ may further include thinned or otherwise weakened areas which may be more susceptible to the expansive forces imposed by the waste. In this manner, expansion of expandable wall 24j′ may be controlled. In a further aspect of the invention, the entirety of receptacle 24′ may be fabricated of elastic material, with those portions of receptacle 24′ being sufficiently thinned and/or weakened so as to define expandable wall 24j′ as described above.

As further seen in FIG. 8, receptacle 24′ may be asymmetrically shaped about a horizontal axis x′-x′ (relative to vertically standing user) extending through the center of opening 14 which is substantially concentrically placed over the user's stoma similar to receptacle 24. The receptacle smaller cavity portion 24f′ is located above axis x′-x′ and the stoma (toward user's head) and the enlarged cavity portion 24e′ is located below axis x′-x′ and the stoma (toward the user's feet). This asymmetrical shape helps minimize the overall size of the receptacle in that the smaller cavity portion 24f′ is located above the stoma where waste does not usually get directed and the enlarged cavity portion 24e′ and expandable wall 24j′ are located below the stoma where the waste and bag 20 are directed within the confines of the receptacle 24′ due to gravity when the user is sitting upright or standing. In this manner, receptacle 24′ may generally expand horizontally in a plane parallel to axis x′-x′ while experiencing little to no expansion vertically (normal to axis x′-x′).

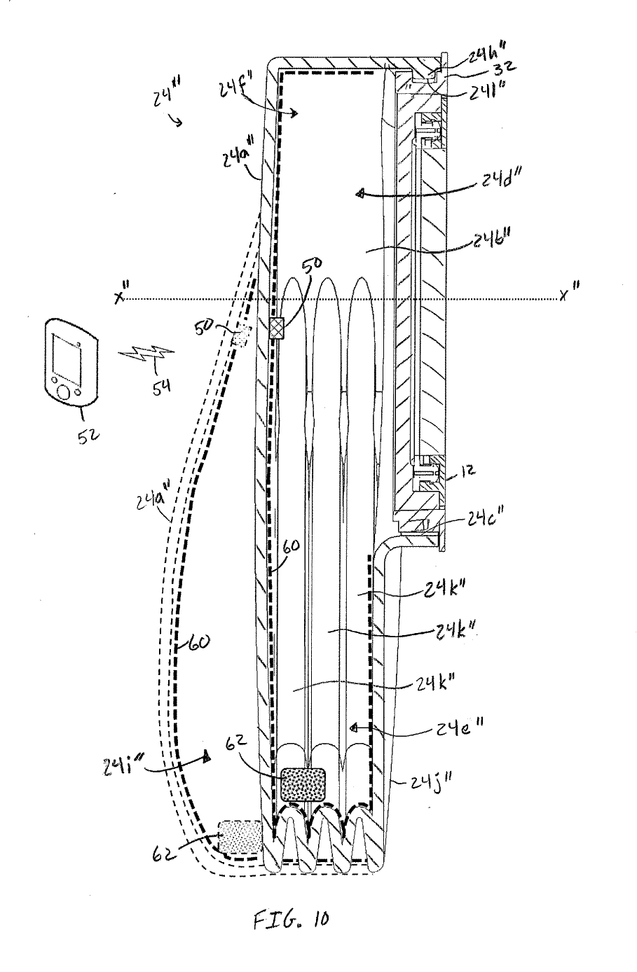

With reference to FIGS. 9 and 10, an alternative cup-shaped receptacle 24″ may be provided for times when the user desires a still greater increased level of discreteness. In accordance with an aspect of the present invention, receptacle 24″ may be a rigid yet flexible member fabricated from any suitable material, such as but not limited to a polymeric material such as polyethylene, polypropylene or nylon, a synthetic or natural rubber or combinations thereof. It should be noted that while latex may be a suitable material for receptacle 24″, care should be taken due to the potential for latex allergies. To that end, receptacle 24″ includes a bottom wall 24a″, a side wall 24b″ and a top wall 24j″ defining a top perimeter edge 24c″ whereby receptacle 24″ defines an interior cavity 24d″ as described above with regard to receptacles 24/24′. Also as described above, the shape of the receptacle is such that side wall 24b″ forms an enlarged cavity portion 24e″ which extends downwardly in line with the length of the body toward the legs when attached to the user in the intended manner. This enlarged cavity portion 24e″ of the receptacle provides more space within which the bag 20 may fit and begin to expand or unroll as the bag fills with the user's waste.

Side wall 24b″ may be constructed to expand/contract through one or more accordion-style folds 24k″ defined along at least a portion of side wall 24b″. Alternatively, side wall 24b″ may be constructed of a resilient elastomeric material configured to expand and retract as described in greater detail below. To that end, bottom wall 24a″ and top wall 24j″ may each be configured to have a higher flexural modulus than side wall 24b″, and more particularly, folds 24k″. In this manner, receptacle 24″ may generally expand horizontally in plane parallel to axis x″-x″ while experiencing little to no expansion vertically (normal to axis x″-x″). Bottom wall 24a″ and top wall 24j″ may thus promote receptacle stability and durability while also assisting the retention of receptacle 24″ onto outer ring 32. With receptacle 24″ in its resting state, bottom wall 24a″ may line in a plane generally parallel with the plane of top wall 24j″. As bag 20 is filled side wall 24b″ is configured to expand outwardly as shown by dashed lines in FIG. 10 so as to form expandable cavity portion 24i″. In accordance with an aspect of the invention, side wall 24b″, and particularly folds 24k″, may be comprised of thinner material which may be more susceptible to the expansive forces imposed upon receptacle 24″ by the waste within bag 20.

As further seen in FIG. 10, receptacle 24″ may be asymmetrically shaped about a horizontal axis x″-x″ (relative to vertically standing user) extending through the center of opening 241″ defined by top perimeter edge 24c″. The receptacle smaller cavity portion 24f″ is located above axis x″-x″ and the stoma (toward user's head) and the enlarged cavity portion 24e″ is located below axis x″-x″ and the stoma (toward the user's feet). This asymmetrical shape helps minimize the overall size of the receptacle in that the smaller cavity portion 24f″ is located above the stoma where waste does not usually get directed and the enlarged cavity portion 24e″ and expandable cavity portion 24i″ are located below the stoma where the waste and bag 20 are directed within the confines of the receptacle 24″ due to gravity when the user is sitting upright or standing.

In a further aspect of the present invention, receptacle bottom walls 24a/24a′/24a″ may include a respective small aperture 24g/24g′/24g″ configured to allow gases from the stoma to vent therethrough. A vent plug 25 may be provided within small aperture 24g/24g′/24g″ whereby vent plug 25 is operable to move between a normally closed aperture position and an open aperture position such that a rise in gas pressure within receptacle 24/24′/24″ above a threshold pressure causes vent plug 25 to move to the open aperture position and release the gas from receptacle 24/24′/24″ through aperture 24g/24g′/24g″. Once the gas has been released and the receptacle internal pressure lowers below the threshold pressure, vent plug 25 may automatically sit back over and close aperture 24g/24g/24g″. Thus, vent 25 allows intermittent gas release rather than constant gas release and also prevents an unsafe pressure build-up within the receptacle.

In accordance with another aspect of the present invention, a receptacle attachment mechanism may be provided for releasably securing receptacle 24/24′/24″ to the disc 12/flange 22 combination. The receptacle attachment mechanism may be provided in the form of a luer-type mechanism, for example. In this preferred embodiment, receptacle top perimeter edge 24c/24c′/24c″ includes one or more tabs 24h/24h′/24h″ which align with and may be screwed into a respective number of curved slots 30 formed in outer ring 32 affixed to disc 12 and thereby releasably securing receptacle 24/24/24′ to outer ring 32. Prior to securing receptacle 24/24′/24″ to ring 32, the user rolls bag 20 up upon itself, such as shown in FIG. 5. Once receptacle 24/24′/24″ is attached to outer ring 32 in the manner described, bag 20 is located within receptacle 24 (FIG. 6), receptacle 24′ (FIG. 8) or receptacle 24″ (FIG. 10). Although a luer-type mechanism is described and shown herein as comprising the receptacle attachment mechanism, it is of course understood that other attachment mechanisms which releasably secure receptacle 24/24′/24″ to outer ring 32 as desired.

In a further aspect, slots 30 formed in outer ring 32 may be configured to provide a two-stage attachment of respective receptacle 24/24′/24″. That is, respective tabs 24h/24h′/24h″ may be inserted within respective slots 30 whereby receptacle 24/24′/24″ may be partially rotated within slot 30 to a first position so as to loosely mount the receptacle against outer ring 32 (first stage). Further rotation of receptacle 24/24′/24″ to a second position may operate to lock receptacle 24/24′/24″ to outer ring 32 in a generally airtight seal (second stage). By way of example, the first stage may be indicated through one or more tabs experiencing a resistance to rotation within its respective slot 30, whereby the second stage may be indicated by tabs 24h/24h′/24h″ to overcoming the resistance to lockingly seat the receptacle against the outer ring. Thereafter, if the pressure within receptacle 24/24′/24″ becomes discernible to the user but has not reached the minimum pressure required to trigger opening of vent 25, the user may manually reverse rotate receptacle 24/24′/24″ to place the receptacle in first stage so as to expel built-up gasses within receptacle 24/24′/24″ without completely removing receptacle 24/24′/24″ from outer ring 32. After release, the receptacle may be rotated to return the receptacle to the second stage as described above. By way of example, should a user by dining at a restaurant and feel pressure within receptacle 24/24′/24″, the user may excuse him/herself from the table and discretely release the gas without expelling the odor at the table. The user may be further comforted knowing that bag 20 will remain secured within receptacle 24/24′/24″ because receptacle 24/24′/24″ remains mounted to outer ring 32 within the first stage and does not need to be completely removed from outer ring 32 to release any gasses.

As described above, waste is free to enter bag 20 which may expand and unroll in the direction of enlarged cavity portion 24e/24e′/24e″ of respective receptacles 24/24e′/24e″. Bag 20 may further expand within expandable cavity portion 24i′ of receptacle 24′ as shown and described with reference to FIG. 8 and expandable cavity portion 24i″ of receptacle 24″ as shown and described with reference to FIG. 10. In any case, there is little to no positive pressure applied to the stoma during this time.

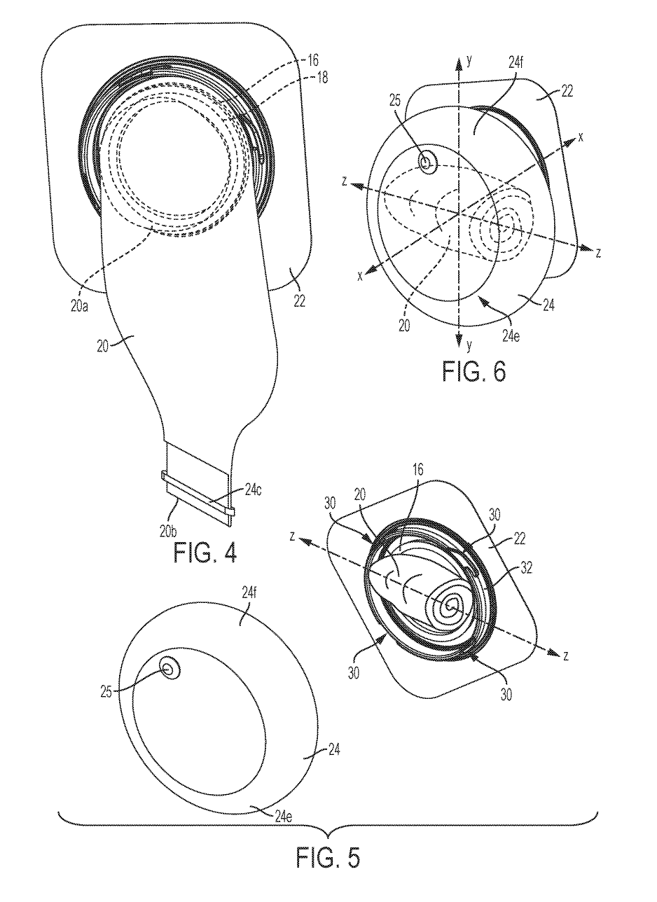

The user may also cover receptacle 24/24′/24″ with a fabric covering 40 having an elasticized opening 40a, see e.g., FIGS. 2 and 8. In this manner, the user may increase his/her level of discreteness by confining the ostomy bag 20 to a relatively small receptacle 24/24′/24″ with a soft fabric covering. The user may thus engage in physical or other social activities with a greater sense of discreteness due to the control and concealment of the ostomy bag afforded by receptacle 24/24′/24″. When the user removes receptacle 24/24′/24″, bag 20 is free to fully unroll whereupon the user may empty waste from second bag opening 20b using resealable closure 20c (see FIG. 4).

In another embodiment, bag 20 may not be used and waste may collect directly in receptacle 24/24′/24″ and will drop by gravity into enlarged cavity portion 24e/24e′/24e″. The collected waste may further expand expandable wall 24j′ of receptacle 24′ or folds 24k′ of receptacle 24″. Receptacle 24/24′/24″ may thereafter be removed, emptied of waste, cleaned and reattached to disc 12, or replaced with a new receptacle 24/24′/24″, as desired. This provides an option to the user who may not want to use a bag or when their bag supply has run out.

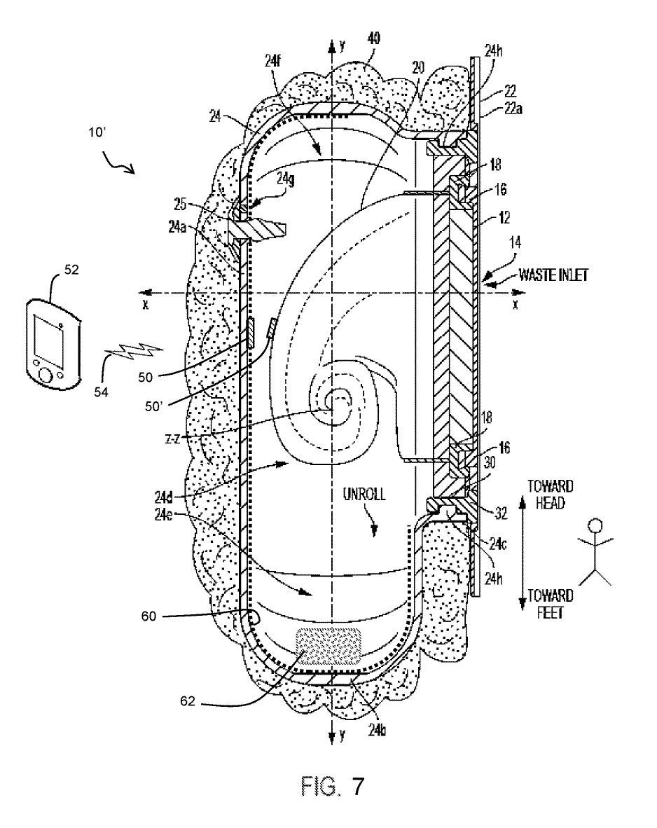

As can be seen in FIGS. 7, 8 and 10, further embodiments of respective receptacles 24/24′/24″ may include a sensor 50 configured to sense when bag 20 (see e.g., FIG. 2) and/or receptacle 24/24′/24″ is full or near full so as to require emptying or changing of bag 20. Sensor 50 may be, for instance and without specifically limited thereto, a pressure sensor that detects pressure upon the sensor as bag 20 expands within receptacle 24/24′/24″ as it is being filled. Placing sensor 50 at or near horizontal axis x-x, x′-x′, x″-x″ may permit bag 20 to more fully occupy respective interior cavity 24e/24e′/24e″, enlarged cavity portion 24f/24f′/24f″ and expandable cavity portion 24i′/24i″ before pressure is exerted upon sensor 50. In this manner, an optimal capacity of bag 20 may be utilized before the filled bag needs to be emptied and/or replaced. Sensor 50 may further include circuitry and software needed to provide a wireless signal 54 to an external device, such as a cell phone 52. Wireless signal 54 may then discretely inform the user of the need to empty and/or replace bag 20. In an alternative embodiment a sensor 50′ may be located on the bag itself such that as the bag fills, the sensor is triggered so as to emit wireless signal 54. It should be noted that while sensors 50/50′ have been described as a pressure sensor, any suitable sensor may be utilized and such other and additional sensors are to be considered within the present disclosure.

In accordance with another embodiment and as seen in FIGS. 7, 8 and 10, respective receptacles 24/24′/24″ may further include a liquid absorbent material to prevent liquid spills or collect liquid should a spill occur. In one aspect, receptacle 24/24′/24″ may be lined with a liquid absorbent paper 60. One possible example of a suitable paper would be from IMPAK Corporation, Los Angeles, Calif., including but not limited to its Grade 460 paper comprised of silica gel impregnated cellulose fibers. Additionally or alternatively, receptacle 24/24′/24″ may be configured to include a liquid absorbent desiccant material—either a loose powder or a powder housed within a liquid dissolvable packet, such as packet 62. One possible example of a suitable packet would be IMPAK Corporation's Liquasafe packets comprising sodium polyacrylate which can absorb up to 300 times its weight in aqueous solutions. It should be understood by those skilled in the art that, while shown and described as being placed within receptacle 24/24′/24″, the desiccant may also be provided within bag 20.

Other embodiments of the invention may include features such as making receptacle 24/24′/24″ of a color changing material which is activated with increased heat. When waste is depositing into the bag or directly into the receptacle, the heat of the waste will heat the receptacle which in turn will change color, altering the user that it may be time to check their ostomy bag or receptacle or possible emptying or changing.

What is claimed is:

1. An ostomy device, comprising:
a) a disc having an adhesive surface for applying to stoma tissue;
b) a ring-shaped flange attached to said disc on the surface thereof opposite said adhesive surface;
c) a receptacle having an interior cavity defined by a bottom wall, a side wall and a top wall having a top perimeter edge, said receptacle being asymmetrically shaped about a horizontal axis relative to a standing user, said asymmetrically shaped receptacle defining an enlarged cavity portion configured to hold a partially filled bag, and wherein at least one of said bottom wall, said side wall and said top wall is fabricated from a rigid yet flexible material;
d) a receptacle releasable attachment mechanism configured to releasably attach said receptacle to said disc.

2. The ostomy device of claim 1 and further including an outer ring affixed to said disc and wherein said releasable attachment mechanism comprises one or more tabs formed on said receptacle top perimeter edge which may releasably attach to a like number of curved slots formed in said outer ring.

3. The ostomy device of claim 2 wherein said releasable attachment mechanism is a two-stage mechanism wherein said one or more tabs formed on said receptacle top perimeter edge is loosely coupled to said curved slots formed in said outer ring in a first stage, and wherein said one or more tabs formed on said receptacle top perimeter edge is lockingly coupled to said curved slots formed in said outer ring in a second stage.

4. The ostomy device of claim 3 wherein said receptacle forms an airtight seal with said outer ring when said releasable attachment mechanism is in said second stage.

5. The ostomy device of claim 1 wherein said receptacle includes an expandable portion configured to expand said enlarged cavity portion when under pressure to thereby define an expandable cavity portion.

6. The ostomy device of claim 5 wherein said expandable portion is comprised of said bottom wall including an expandable wall configured to expand said enlarged cavity portion parallel to said horizontal axis.

7. The ostomy device of claim 5 wherein said expandable portion is defined of said side wall whereby said side wall is configured to expand said enlarged cavity portion parallel to said horizontal axis.

8. The ostomy device of claim 7 wherein said side wall is fabricated of an elastomeric material configured to expand said enlarged cavity portion when under pressure.

9. The ostomy device of claim 7 wherein said side wall includes one or more fold configured to expand said enlarged cavity portion when under pressure.

10. The ostomy device of claim 1 and further including an aperture in said receptacle bottom wall and a vent plug located within said aperture and operable to move between a normally closed aperture position and an open aperture position where a rise in pressure of a gas within said receptacle above a threshold pressure causes said vent plug to move to said open aperture position to thereby release said gas from said receptacle through said aperture.

11. The ostomy device of claim 1 wherein said receptacle includes a color changing material which is activated by heat.

12. The ostomy device of claim 1 and further comprising:
e) a bag having a first opening having a ring-shaped groove attached to the perimeter of said first opening, said ring-shaped groove releasably attachable to said ring-shaped flange to releasably attach said bag to said disc.

13. The ostomy device of claim 1 wherein an internal surface of said bottom wall of said receptacle includes a sensor opposite said top perimeter edge, the sensor configured to communicate when said receptacle is full or near full.

14. The ostomy device of claim 13 wherein said sensor is a pressure sensor.

15. The ostomy device of claim 13 wherein said sensor emits a wireless signal configured to be received by an external device.

16. The ostomy device of claim 12 wherein said bag includes a sensor opposite the first opening, the sensor configured to communicate when said bag is full.

17. The ostomy device of claim 1 wherein an internal surface of said receptacle is lined with a liquid absorbent paper.

18. The ostomy device of claim 1 wherein the receptacle includes liquid absorbent desiccant material within said enlarged cavity portion.

19. The ostomy device of claim 12 wherein said bag is constructed of a biodegradable material.

20. The ostomy device of claim 12 wherein said bag is constructed of a flushable material.

21. A method of using an ostomy appliance, said method comprising the steps of:
a) providing an ostomy flange having a disc and an adhesive surface and a central opening;
b) releasably attaching said disc to a user via said adhesive surface with said central opening located over the user's stoma;
c) providing an asymmetrical receptacle having a bottom wall, side wall and open perimeter top all defining an interior cavity having an enlarged cavity portion wherein the receptacle is asymmetrically shaped about a horizontal axis relative to a standing user; and
d) removably attaching said receptacle to said disc with said open top perimeter positioned over said disc central opening and stoma.

22. The method of claim 21 and further comprising the step of orienting said receptacle with said enlarged cavity portion extending in the direction of the user's feet.

23. The method of claim 21 and further comprising:
e) providing a bag having an open top; and
f) prior to removably attaching said receptacle to said disc, removably attaching said bag to said disc with said bag top opening positioned over said central opening and stoma.

24. The method of claim 23 and further comprising:
g) prior to removably attaching said receptacle to said disc, rolling said bag up on itself about an axis which extends perpendicular to the longitudinal axis of said receptacle.

25. The method of claim 21 and further comprising:
e) providing an outer ring with one or more curved slots formed therein and affixing said outer ring to said disc;
f) providing one or more tabs on said receptacle top perimeter edge; and
g) releasably attaching said receptacle to said outer ring with said one or more tabs engaging said one or more curved slots, respectively.

26. The method of claim 21 wherein step (g) further comprises:
i) rotating said receptacle to a first stage wherein each of said one or more tabs on said receptacle top perimeter edge is loosely coupled to each of said respective curved slots formed in said outer ring; and
ii) further rotating said receptacle to a second stage wherein each of said one or more tabs on said receptacle top perimeter edge is lockingly coupled to said curved slots formed in said outer ring.
